(19) United States
(12) Patent Application Publication
(10) Pub. No.: US 2015/0297552 A1
(43) Pub. Date: Oct. 22, 2015

(54) FERTILITY ENHANCING AGENT

(71) Applicant: OCTAPAM LIMITED, Lamaca (CY)

(72) Inventors: Rahimdzhan Ahmetdzhanovich ROZIEV, Obninsk (RU); Anna Yakovlevna GONCHAROVA, Obninsk (RU); llya Vladimirovich VOROBYEV, Obninsk (RU); Kenes Tagayevich ERIMBETOV, Borovsk (RU); Vladimir Konstantinovich PODGORODNICHENKO, Obninsk (RU)

(21) Appl. No.: 14/386,009

(22) PCT Filed: Mar. 20, 2013
(86) PCT No.: PCT/IB2013/001124

§ 371 Date: Sep. 18, 2014

(30) Foreign Application Priority Data
Mar. 21, 2012 (RU) 2012110568

(51) Int. Cl. A61K 31/33; A61K 47/48

(52) U.S. Cl. CPC A61K 31/33; A61K 47/48969

(57) ABSTRACT

Present invention relates to the field of medicine, in particular, to adrology and is also useful in veterinary medicine for treating mammals having impaired fertility. The invention provides a fertility enhancing drug, wherein the drug is a clathrate complex of β-cyclodextrine with 9-phenyl-sym-octahydroselenoxanthene in α-crystalline form represented by structural formula. Technical effect provided by present invention consists in improvement of pregnancy and fertility parameters. 6 tables.

### RELATED APPLICATIONS

This application claims priority to Patent Cooperation Treaty Application number PCT/IB2013/001124, filed on Mar. 20, 2013, which claims priority to Russian patent application numbers RU2012110568, filed on Mar. 21, 2012, and incorporated herewith by reference in its entirety.

### FIELD OF THE INVENTION

Present invention relates to the field of medicine, in particular, to adrology and further is useful in veterinary medicine for treating mammals having impaired fertility.

### BACKGROUND OF THE INVENTION

Infertility is a serious worldwide health and socioeconomic problem. According to different studies, 15 to 25 percent of married couples are currently diagnosed as infertile and these figures tend to increase. Approximately 40% of conception failure in a marriage are attributed to either male or female infertility, and approximately 20% of cases result from infertility of both partners. Causes of male infertility leading to impairment of normal spermatogenesis and abnormality of reproductive hormones secretion may considerably vary. Depending on the pathological process underlying pathological semen, surgical or drug-based methods are used to treat male infertility.

Restoration of reproductive health of a man having impaired fertility using available drugs faces serious problems connected with absence of drugs providing sustained and effective stimulation and/or restoration of spermatogenesis. Generally, treatment requires use of a drug complex including antibacterial and anti-inflammatory agents together with hormonal drugs which leads to dystrophic changes in seminiferous tubules and interstitium of testicles. In this situation, a practitioner have to rely on the capability of a man's body to restore the spermatogenesis after elimination of the underlying pathological process. However, this is by no means always the case. Besides infectious agents and endogenous factors, the problem of exogenous intoxications has gained special significance during the last decade. This problem is connected with environmental accumulation of high amounts of chemicals having deteriorative effect on a men's reproductive system. Similarly, uncontrolled use of drugs such as sulfonylamides, antibiotics, cytostatic agents etc. has deteriorative effect of spermatogenous (germinal) epithelium of testicles.

The above listed endogenous factors and additionally a number of occupational intoxications lead to severe oxidative stress. It was found that approximately 40% of infertile men accumulate free radicals in their reproductive system, the radicals being capable of affecting the highly sensitive germinal epithelium of testicles.

Connection between selenium and impairment of reproductive function in animals was found some time ago and considerable amount of studies has been devoted to this problem. In animals, deficiency of dietary selenium leads to multiple metabolic changes similar to vitamin A: deficiency: growth stunt, degenerative and dystrophic changes of skeletal muscles, cardiac muscle, neurons, liver, lungs, and other organs. Animal and birds of different species and of either sex are affected by reproductive function impairments such as: ovarian cycle disruption, increased embryonic mortality, high infertility percentage, [symbol] , decreased libido, decreased amount and quality of sperm. Selenium deficiency leads to breakage of a sperm in the mid-tail region where a selenopeptide was found, this selenopeptide playing an important role in the tail formation process. Further, this selenopeptide is a structural protein of mitochondria and enzymes within the sperm head and comprises a seminal plasma component.

A drug for treating sexual function in men is disclosed in RU2163806 (A61K31/475, 2001). This drug comprises yohimbine hydrochloride, ginseng root, sodium selenite, zinc oxide, and ascorbic acid.

Znc amino acid chelate, selenium, and puregon have been used in combination with physiotherapeutic treatment to improve sperm quality in individuals having pathological semen. See RU2205047 (A61N5/067, 2001).

RU2249450 (A61K31/00, 2005) discloses use of a prolonged release dosage form of selenopyrane in oil (prolonged dosage form) concurrently with E, D, and A vitamins to improve reproductive function in bulls.

Each of the above agents comprises either selenium or a selenium-comprising compound. Selenium and compounds thereof are known to be toxic and due to this toxicity selenium cannot be used in amounts sufficient to attain the desired effect.

Russian patent RU2414215 (A61K31/095, 2011) is the closest prior art relating to the technical essence of the present invention. RU2414215 discloses a drug for improving reproductive function, wherein the drug is α-crystalline form of 9-phenyl-sym-octahydroselenoxanthene of structural formula

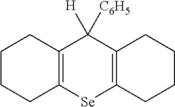

and characterized by powder X-ray diffraction pattern obtained using Cu—K radiation emitter and having characteristic diffraction angles 2θ: 6.0, 12.0, 15.0, 17.0, 19.0, 20.0, 21.5, 21.7, 20.9, 25.0, 27.0, 28.0, 29.0, 37.0, and by melting temperature 96.8° C.

However, the prototype drug does not provide the desired pregnancy and fertility parameters.

The object of the present invention is to provide an agent devoid of said limitations.

Therefore, the technical effect of the present invention consists in improvement of pregnancy and fertility parameters.

### SUMMARY OF THE INVENTION

According to the invention, said problem is solved to attain this technical effect by providing a fertility enhancing agent, wherein the agent is a clathrate complex of β-cyclodextrine with 9-phenyl-sym-octahydroselenoxanthene represented by structural formula

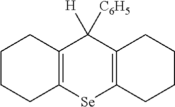

Said 9-phenyl-sym-octahydroselenoxanthene compound can be provided in α-crystalline form characterized by powder X-ray diffraction pattern obtained using Cu—K radiation emitter and having characteristic diffraction angles 2θ: 6.0, 12.0, 15.0, 17.0, 19.0, 20.0, 21.5, 21.7, 20.9, 25.0, 27.0, 28.0, 29.0, 37.0 and by 100 melting temperature of 96.8° C.

Providing 9-phenyl-sym-octahydroselenoxanthene in a clathrate form result in a agent having activating effect on functions of reproductive organs thus attaining the technical effect.

### DETAILED DESCRIPTION OF THE PREFERRED EMBODIMENT

Improvement of reproductive function in rats treated by the agent has been demonstrated in two independent experiments. The present invention is further explained by specific embodiement.

### Example 1

180 male and female Wistar rats were tested in the 110 experiments (initial body weight—180-200 g).

Group of 60 male rats was grouped into 6 subgroups each including 10 animals. Each group was treated as follows: group 1—vegetable oil, group 2—β-cyclodextrin, group 3—the agent of present invention, 0.35 mg/kg (the prototype agent), group 4—medication, 0.70 mg/kg (the prototype agent), group 5—the agent of present invention in a clathrate complex, 0.35 mg/kg, group 6—the agent of present invention in a clathrate complex, 0.70 mg/kg.

Male rats of the study subgroups were intragastrically given the above mentioned doses of the prototype agent and the agent of present invention in the clathrate complex form daily during 10 weeks (2-3 cycles of spermatozoa maturation). At the same time, animals of control groups were treated with corresponding amounts of placebo consisting of the vegetable oil or β-cyclodextrin. The placebo was chosen based on the fact that the prototype agent is an oil-soluble compound and the agent of present invention in the clathrate complex form is a water soluble compound. The drugs were administered once daily (at 10 a.m.).

After completion of dosing 6 groups of animals were formed:
the first (vegetable oil)—20 control female rats were housed together with 10 control male rats; the second (β-cyclodextrin)—20 control female rats were housed together with 10 control male rats; the third (the prototype agent, 0.35 mg/kg)—20 study female rats housed together with 10 control male rats;
the fourth (the prototype, 0.70 mg/kg)—20 control female rats were housed together with 10 study male rats;
the fifth (the agent of present invention in the clathrate complex form 0.35 mg/kg)—20 study female rats were housed together with 10 control male rats;
the sixth (the agent of present invention in the clathrate complex form 0.70 mg/kg)—20 control female rats were housed together with 10 study male rats.

During two estrous cycles, vaginal smear preparations were analyzed. Proestrus, estrus and metaestrum phases were detected. Presence of spermatozoa in vaginal smear preparations was registered as the first day of gestation. After 10 days, two subgroups of gestation female rats were formed from female rats in each of five groups. Female rats of the first subgroups were sacrificed on 20^{th} day of gestation. Amount of fetuses, resorption, implantation sites in the uterine cavity and yellow bodies of ovary were counted. Based on the obtained data, pre-implantation and post-implantation fetal death indexes were calculated according to the following equation:

$$Pre \text{-} implantation death , \% = \frac{( {N}_{1} - {N}_{2} ) \times 100}{{N}_{1}} ,$$

where
N₁—amount of yellow bodies,
N₂—amount of implantation sites

$$Post \text{-} implantation death , \% = \frac{( {N}_{1} - {N}_{2} ) \times 100}{{N}_{1}} ,$$

where
N₁—amount of implantation sites
N₂—amount of viable fetuses.

Further, fertility index (ratio between the amount of female rats housed together with male rats to the amount of female rats having spermatozoa in vaginal smear preparations) and gestation index (ratio between the amount of female rats having spermatozoa in vaginal smear preparations to the amount of animals with full-term pregnancy):

$$Fertility index , \% = \frac{{N}_{1} \times 100}{{N}_{2}} ,$$

where
N₁—amount of embryonate female rats
N₂—amount female rats housed together with male rats

$$Gestation index , \% = \frac{{N}_{1} \times 100}{{N}_{2}} ,$$

where
N₁—amount of gestation female rats
N₂—amount of embryonate female rats
Pathological changes (subcutaneous hematomas and edemas, developmental defects of skeletal system and eye globes) were registered.

The second groups of gestation female rats were allowed to parturiate under monitoring. Animals were weighed weekly and weight gain was registered. Parturition date, amount of babies in the brood and weight of babies were recorded. On 21^{st} day after parturition, the survival rate and total and median weight of babies were measured.

TABLE 1
Effect of the prototype agent and the the agent of present invention in the clathrate complex
form administered to male rats in doses of 0.35 and 0.70 mg/kg on fertility index

| | Groups of animals | | | | | |
|---|---|---|---|---|---|---|
| | | | | | 5 | 6 |
| | | | | | Agent of | Agent of |
| | | | 3 | 4 | present invention | present invention |
| | | | Prototype | Prototype | in the clathrate | in the clathrate |
| | 1 | 2 | agent, | agent, | complex form * | complex form * |
| Tested indexes | Vegetable oil | β-cyclodextrin | 0.35 mg/kg | 0.70 mg/kg | 0.35 mg/kg | 0.70 mg/kg |
| Amount female rats | 20 | 20 | 20 | 20 | 20 | 20 |
| housed together with | | | | | | |
| males | | | | | | |
| Amount of embryonate | 13 | 14 | 16 | 17 | 19 | 18 |
| female rats | | | | | | |
| Amount of gestation | 10 | 11 | 13 | 14 | 18 | 16 |
| female rats | | | | | | |
| Fertility index, %. | 65 | 70 | 80 | 85 | 95 | 90 |
| Gestation index, %. | 77 | 79 | 81 | 82 | 95 | 89 |

Remark:
* Agent of present invention in the clathrate complex with β-cyclodextrin.

TABLE 2
Effect of the Prototype agent and the Agent of present invention in the clathrate complex form administered
to male rats in doses of 0.35 and 0.70 mg/kg on fertility characteristics of the rats

| | Groups of animals | | | | | |
|---|---|---|---|---|---|---|
| | | | | | 5 | 6 |
| | | | | | Agent of | Agent of |
| | | | 3 | 4 | present invention | present invention |
| | | | Prototype | Prototype | in the clathrate | in the clathrate |
| | 1 | 2 | agent | agent | complex form ** | complex form ** |
| Tested characteristics | Vegetable oil | β-cyclodextrin | 0.35 mg/kg | 0.70 mg/kg | 0.35 mg/kg | 0.70 mg/kg |
| Amount of gestation female rats | 10 | 11 | 13 | 14 | 18 | 16 |
| Amount of yellow bodies | 10.5 ± 0.6 | 10.8 ± 0.8 | 13.8 ± 1.2* | 13.2 ± 0.9* | 14.2 ± 1.3* | 13.8 ± 1.2* |
| Amount of implantation sites | 9.8 ± 0.8 | 10.1 ± 0.7 | 13.1 ± 1.0* | 12.5 ± 1.0* | 13.7 ± 1.2* | 13.2 ± 1.0* |
| Amount of viable fetuses | 9.1 ± 1.0 | 9.4 ± 0.7 | 12.4 ± 0.8* | 11.9 ± 0.7* | 13.2 ± 1.6* | 12.,6 ± 1.0* |
| Amount of dead fetuses | 0 | 0 | 0 | 0 | 0 | 0 |
| Amount of resorptions | 0.5 ± 0.2 | 0.5 ± 0.1 | 0.6 ± 0.2 | 0.5 ± 0.2 | 0.4 ± 0.1 | 0.4 ± 0.2 |
| Pre-implantation loss, % | 6.7 | 6.5 | 5.1 | 5.3 | 3.5 | 4.3 |
| Post-implantation loss, % | 7.1 | 6.9 | 5.3 | 4.8 | 3.6 | 5.4 |
| Fetus weight, g | 3.2 ± 0.1 | 3.1 ± 0.2 | 3.3 ± 0.2 | 3.3 ± 0.1 | 3.6 ± 0.1* | 3.5 ± 0.2 |
| Craniocaudal size of fetus, mm | 32 ± 2 | 31 ± 1 | 33 ± 2 | 33 ± 1 | 36 ± 1* | 35 ± 2 |

Remark:
*hereinafter P < 0.05 in comparison to animals of control groups
** Agent of present invention in the clathrate complex with β-cyclodextrin

TABLE 3
Effect of the prototype agent and the medication as a Agent of present invention in the clathrate complex
administered to male rats in doses of 0.35 and 0.70 mg/kg on postnatal development of rat babies

| | Groups of animals | | | | | |
|---|---|---|---|---|---|---|
| | | | | | 5 | 6 |
| | | | | | Agent of | Agent of |
| | | | 3 | 4 | present invention | present invention |
| | | | Prototype | Prototype | in the clathrate | in the clathrate |
| | 1 | 2 | agent | agent | complex form ** | complex form ** |
| Study characteristics | Vegetable oil | β-cyclodextrin | 0.35 mg/kg | 0.70 mg/kg | 0.35 mg/kg | 0.70 mg/kg |
| Amount of babies delivered | 8.0 ± 0.6 | 8.1 ± 0.5 | 9.2 ± 1.1 | 9.0 ± 1.2 | 9.7 ± 0.4* | 9.4 ± 1.1 |
| by 1 female | | | | | | |
| Postnatal loss after 3 weeks, % | 3.4 | 3.5 | 2.8 | 2.8 | 1.8 | 2.0 |
| Weight of newborn baby | 5.2 ± 2.9 | 5.1 ± 1.8 | 5.7 ± 0.9 | 5.8 ± 1.0 | 6.2 ± 0.8 | 6.1 ± 0.9 |
| rats, g | | | | | | |
| Dynamics of babies' body | | | | | | |
| weight, g | | | | | | |
| 1^{st} week | 16.5 ± 1.2 | 16.7 ± 1.0 | 19.2 ± 1.1 | 19.1 ± 0.8 | 19.9 ± 0.9* | 19.5 ± 1.0 |
| 2^{nd} week | 25.5 ± 2.8 | 25.2 ± 1.5 | 25.2 ± 1.6 | 25.3 ± 1.2 | 27.2 ± 1.1 | 26.8 ± 1.3 |
| 3^{rd} week | 42.8 ± 1.9 | 43.3 ± 2.3 | 45.7 ± 1.2 | 44.7 ± 1.5 | 48.0 ± 1.3 | 47.9 ± 1.4 |
| 4^{th} week | 57.6 ± 2.7 | 57.2 ± 2.9 | 60.4 ± 1.9 | 59.8 ± 2.4 | 62.7 ± 1.7 | 61.9 ± 2.0 |

Remark:
** Agent of present invention in the clathrate complex with β-cyclodextrin

Analysis of rat fertility indexes (Table 1) revealed increase of said indexes in groups 3, 4, 5 and 6 (study) in comparison to indexes of control groups 1 and 2. More significant increase of fertility indexes was observed after administiration of the agent of present invention in the clathrate complex form.

Intragastrical administration of the agent of present invention in the clathrate complex form to male rats in amount of 0.35 and 0.70 mg/kg lead to statistically significant increase in female rats fertility parameters such as amounts of yellow bodies, implantation sites, viable fetuses, and did effect resorption rates. Pre-implantation and post-implantation loss in groups 1 and 2 group were higher than as compared to study groups. Embryo body weight and craniocaudal size were significantly increased after administration of the agent of present invention in the clathrate complex form to male rats in amount of 0.35 and 0.70 mg/kg as compared to groups 1 and 2 (Table 2).

Weight gain of gestation rats in study groups was higher as compared to control groups. Amount of babies delivered by 1 rat, weight of newborn babies and further weight gain of newborn babies during the first month of neonatal development were also higher in all 4 study groups as compared to control groups. At the same time, the significant increase of said parameters was observed after treatment with the agent of present invention in the clathrate complex form in amount of 0.35 mg/kg (Table 3).

Physical development of babies during the first month of postnatal development (hair growing, appearance of incisor teeth, eye opening, pinna detachment, vaginal opening, testes descent etc.) did not deviate from the timeline characteristic for normal physiological development of animals of this species.

As a result of conducted experiments, it was found that oral administration of the agent of present invention to male rats in amount of 0.35 and 0.70 mg/kg has positive effect on animal reproductive function. At the same time, reproductive parameters such as amount of viable fetuses, amount of yellow bodies, amount of implantation sites, body weight and craniocaudal size of fetuses were increased, pre- and post-implantation losses were decreased and amount of resorptions remained unchanged. The best resulted were observed after administration of the agent of present invention in the clathrate complex form to male rats in amount of 0.35 mg/kg.

### Example 2

180 male and female Wistar rats were studied in the experiments (initial body weight—180-200 g). The prototype agent and the agent of present invention in the clathrate complex form were administered intragastrically in amount of 0.35 mg/kg. Female group of 120 animals was broken into two subgroups: control group (80 animals) and study group (40 animals). The prototype agent and the agent of present invention in the clathrate complex form were intragastrically administered to female rats of study group once a day during 2 weeks (3-4 estrous cycles). Male group of 60 animals was broken into two subgroups: control group (40 animals) and study group (20 animals). The prototype agent and the agent of present invention in the clathrate complex form were intragastrically administered to male rats of study group daily during 10 weeks (2-3 cycles of spermatozoa maturation). Corresponding amounts of placebo comprised of vegetable oil or β-cyclodextrin were administered to animals of the control group with the same intervals. The placebo was chosen based on the fact that the prototype agent is an oil-soluble compound and the agent of present invention in the clathrate complex form is a water soluble compound. The drugs were administered once daily (at 10 a.m.).

After administering the medication 6 groups of animals were formed:
the first (vegetable oil)—20 control female rats were housed together with 10 control male rats;
the second (β-cyclodextrin)—20 control female rats were housed together with 10 control male rats;
the third (the prototype, 0.35 mg/kg)—20 study female rats were housed together with 10 control male rats;
the fourth (the prototype, 0.35 mg/kg)—20 control female rats were housed together with 10 study male rats;
the fifth (the agent of present invention in the clathrate complex form 0.35 mg/kg)—20 study female rats were housed together with 10 control male rats;
the sixth (the agent of present invention in the clathrate complex form 0.35 mg/kg)—20 control female rats were housed together with 10 study male rats.

A pre- and post-implantation fetal death indexes, fertility index and gestation index were calculated according to equations cited in Example 1.

TABLE 4
Effect of the prototype agent and agent of present invention in the clathrate complex form when administered
to male rats and female rats separately in amounts of 0.35 mg/kg on rat fertility index

| | Groups of animals | | | | | |
|---|---|---|---|---|---|---|
| | | | | | 5 | 6 |
| | | | | | Agent of | Agent of |
| | | | | | present invention | present invention |
| | | | 3 | 4 | in the clathrate | in the clathrate |
| | | | Prototype | Prototype | complex * with | complex * with |
| | | | agent was | agent was | β-cyclodextrin | β-cyclodextrin |
| | 1 | 2 | administered | administered | was administered | was administered |
| Study characteristics | Vegetable oil | β-cyclodextrin | to female rats | to male rats | to female rats | to male rats |
| Amount of added | 20 | 20 | 20 | 20 | 20 | 20 |
| female rats | | | | | | |
| Amount of | 15 | 14 | 16 | 17 | 18 | 19 |
| embryonate female | | | | | | |
| rats | | | | | | |
| Amount of gestation | 12 | 11 | 14 | 15 | 17 | 18 |
| female rats | | | | | | |
| Fertility index, %. | 75 | 70 | 80 | 85 | 90 | 95 |
| Gestation index, %. | 80 | 79 | 88 | 88 | 94 | 95 |

Remark:
* Agent of present invention in the clathrate complex form with β-cyclodextrin

TABLE 5
Effect of the prototype agent and agent of present invention in the clathrate complex form when administered
to male rats and female rats separately in amounts of 0.35 mg/kg on rat fertility index

| | Groups of animals | | | | | |
|---|---|---|---|---|---|---|
| | | | | | 5 | 6 |
| | | | | | Agent of | Agent of |
| | | | | | present invention | present invention |
| | | | 3 | 4 | in the clathrate | in the clathrate |
| | | | Prototype | Prototype | complex ** with | complex ** with |
| | | | agent was | agent was | β-cyclodextrin | β-cyclodextrin |
| | 1 | 2 | administered | administered | was administered | was administered |
| Studied parameters | Vegetable oil | β-cyclodextrin | to female rats | to male rats | to female rats | to male rats |
| Amount of gestation female | 12 | 11 | 14 | 15 | 17 | 18 |
| rats | | | | | | |
| Amount of yellow bodies | 9.9 ± 0.9 | 10.0 ± 0.8 | 10.7 ± 0.7 | 10.8 ± 0.8 | 12.3 ± 0.6* | 12.5 ± 0.7* |
| Amount of implantation sites | 9.0 ± 0.8 | 9.1 ± 0.7 | 9.9 ± 0.6 | 10.1 ± 1.1 | 11.9 ± 0.7* | 12.0 ± 0.6* |
| Amount of viable fetuses | 8.1 ± 0.9 | 8.2 ± 1.0 | 9.2 ± 1.1 | 9.4 ± 1.0 | 11.2 ± 0.9* | 11.3 ± 1.0* |
| Amount of dead fetuses | 0 | 0 | 0 | 0 | 0 | 0 |
| Amount of resorptions | 0.6 ± 0.2 | 0.5 ± 0.2 | 0.4 ± 0.2 | 0.4 ± 0.2 | 0.3 ± 0.1 | 0.3 ± 0.2 |
| Pre-implantation loss, % | 9.1 | 9.0 | 7.5 | 6.5 | 3.3 | 4.0 |
| Post-implantation loss, % | 10.0 | 9.9 | 7.1 | 6.9 | 5.9 | 5.8 |
| Fetus body weight, g | 3.2 ± 0.2 | 3.1 ± 0.1 | 3.4 ± 0.2 | 3.5 ± 0.2 | 3.6 ± 0.1* | 3.7 ± 0.1* |
| Carniocaudal size of fetus, | 33 ± 1 | 32 ± 1 | 35 ± 2 | 35 ± 1 | 36 ± 1* | 37 ± 2* |
| mm | | | | | | |

Remark:
** Agent of present invention in the clathrate complex form with β-cyclodextrin

TABLE 6
Effect of the prototype agent and agent of present invention in the clathrate complex form when administered
to male rats and female rats separately in amounts of 0.35 mg/kg on rat fertility index

| | Groups of animals | | | | | |
|---|---|---|---|---|---|---|
| | | | | | 5 | 6 |
| | | | | | Agent of | Agent of |
| | | | | | present invention | present invention |
| | | | 3 | 4 | in the clathrate | in the clathrate |
| | | | Prototype | Prototype | complex form** with | complex form** with |
| | | | agent was | agent was | β-cyclodextrin | β-cyclodextrin |
| | 1 | 2 | administered | administered | was administered | was administered |
| Study characteristics | Vegetable oil | β-cyclodextrin | to female rats | to male rats | to female rats | to male rats |
| Amount of babies | 7.9 ± 0.5 | 8.0 ± 0.4 | 8.6 ± 0.9 | 8.7 ± 0.8 | 9.2 ± 0.7 | 9.5 ± 0.4* |
| delivered by 1 female | | | | | | |
| Postnatal loss after 3 | 3.1 | 3.2 | 2.5 | 2.6 | 2.0 | 2.1 |
| weeks, % | | | | | | |
| Weight of newborn | 5.9 ± 0.8 | 5.8 ± 0.6 | 6.2 ± 0.9 | 6.4 ± 1.2 | 6.7 ± 0.8 | 7.0 ± 0.9 |
| baby rats, g | | | | | | |
| Dynamics of infant | | | | | | |
| body weight, g | | | | | | |
| 1st week | 16.8 ± 1.8 | 16.6 ± 1.5 | 18.8 ± 1.2 | 20.1 ± 0.9 | 20.9 ± 1.1 | 21.4 ± 1.0* |
| 2nd week | 24.6 ± 2.3 | 24.4 ± 1.9 | 26.3 ± 1.5 | 27.2 ± 1.5 | 28.1 ± 1.5 | 28.4 ± 1.5 |
| 3rd week | 46.2 ± 1.8 | 45.0 ± 1.4 | 47.4 ± 2.0 | 45.9 ± 1.9 | 47.8 ± 1.7 | 48.0 ± 1.5 |
| 4th week | 58.8 ± 1.4 | 58.3 ± 1.9 | 61.5 ± 2.8 | 61.9 ± 2.6 | 62.8 ± 2.2 | 63.4 ± 1.,8* |

Remark:
**Agent of present invention in the clathrate complex with β-cyclodextrin

Analysis of fertility indexes in rats (Table 4) revealed increase of said indexes in groups 3, 4, 5 and 6 (study) as compared to indexes of control groups 1 and 2. More significant increase of fertility indexes was observed after administration of agent of present invention in the clathrate complex form to male rats in amount of 0.35 mg/kg.

Oral administration of the agent of present invention in the clathrate complex form in amount of 0.35 amg/kg to male rats and female rats caused significant increase in female rats fertility parameters such as amounts of yellow bodies, implantation sites, viable fetuses, and did not affect resorption rates.

Pre-implantation and post-implantation loss in control groups was higher than the loss in study groups. Embryo body weight and craniocaudal size were significantly increased after separate administration of the agent of present invention in the clathrate complex form to male rats and female rats in amount of 0.35 mg/kg as compared to control groups (Table 5).

Weight gain of gestation rats in study groups was higher as compared to control groups. Amount of babies delivered by 1 rat, weight of newborn babies and further weight gain of newborn babies during the first month of neonatal development were also higher in all 4 study groups as compared to control groups. At the same time the reliable increase of said characteristics was observed after administering the agent of present invention in the clathrate complex form in amount of 0.35 mg/kg to male rats (Table 6).

Thus, the most significant influence on rat fertility was observed after administration of the agent of present invention in the clathrate complex form in amount of 0.35 mg/kg to male rats.

1. A fertility enhancing agent, wherein the agent is a clathrate complex of β-cyclodextrine with 9-phenyl-sym-octahydroselenoxanthene represented by structural formula

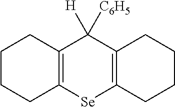

2. The drug of claim 1, wherein the 9-phenyl-sym-octahydroselenoxanthene is in α-crystalline form characterized by X-ray powder diffraction (XRD) pattern as obtained using a Cu—K emitter, the XRD pattern having characteristic diffraction angles 2θ: 6.0, 12.0, 15.0, 17.0, 19.0, 20.0, 21.5, 21.7, 20.9, 25.0, 27.0, 28.0, 29.0, 37.0, and by melting temperature 96.8° C.
